(19) United States
(12) Patent Application Publication
(10) Pub. No.: US 2018/0000551 A1
(43) Pub. Date: Jan. 4, 2018

(54) APPARATUS FOR PROTECTING SURGICAL INSTRUMENT TRANSPORT TRAYS

(71) Applicant: Karim Wahib, New York, NY (US)

(72) Inventor: Karim Wahib, New York, NY (US)

(21) Appl. No.: 15/611,109

(22) Filed: Jun. 1, 2017

Related U.S. Application Data
(60) Provisional application No. 62/344,145, filed on Jun. 1, 2016.

(51) Int. Cl. A61B 50/33; A61L 2/26; B65D 1/34; A61B 50/20

(52) U.S. Cl. CPC A61B 50/33; B65D 1/34; A61L 2/26; A61B 50/20

(57) ABSTRACT

A surgical instrument tray enclosure comprising at least four sidewalls, at least four corners including pillars connected at exterior portion of the at least four corners of the at least four sidewalls and an interior corner having a rounded curve that is tangential to the pillars, the pillars are cylindrical in shape having bulbous top and bottom ends and extend a given distance beyond the bulbous top and bottom ends, and a base at a bottom of the at least four sidewalls to form a cavity where a wrapped surgical instrument tray may be inserted or bound within the cavity.

### COPYRIGHT NOTICE

A portion of the disclosure of this patent document contains material, which is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### BACKGROUND OF THE INVENTION

### Field of the Invention

This application generally relates to a protective enclosure, and in particular, a protective enclosure for surgical instrument transport trays designed to protect surgical instrument trays or pans which have been wrapped in a sterilization wrap from puncture.

### Description of the Related Art

While a number of the hospital and operating room supplies today are disposable, most hospitals still reuse many if not most of the surgical instruments within the operating room. As a result, each time they are used, they must be washed and re-sterilized before they can be used again. The sterilization process typically involves the placement of the surgical instruments within a sterilization unit such as an open-top stainless steel tray which has a perforated bottom. The instrument tray is then wrapped in a material which will allow the entry of the sterilant such as steam or ethylene oxide while prohibiting the entry of bacteria or other contaminants after the sterilization process has been completed. Once the instruments have been sterilized, they are kept within the instrument tray in the wrapped configuration until their actual use during a surgical procedure.

If the barrier provided by the wrapping of the instrument tray is breached at any time prior to the use of the instruments, then the contents are considered contaminated and unusable until the instrument tray has been rewrapped and re-sterilized. The highest probability for breaching these wrapped and sterilized instrument trays is during the transporting and storage of them. Hospitals have been attempting to reduce the cuts, holes and tears in the sterilization wrap by cushioning the bottom of the instrument trays either with a huck towel between the instrument tray and the sterilization wrap or with some form of cushioning on the storage shelves, usually in the form of foam or a used linen. This cushioning reduces the amount of shearing motion that the wrap experiences when a wrapped instrument tray is pulled or dragged across the storage shelf, thus reducing the chance for the occurrence of cuts, holes, and tears in the sterilization wrap.

Current means to reduce breaching of instrument tray wrappings are inadequate in providing protection to instrument trays in repeated occurrences of shearing forces during storage, handling, and transport of the tray. There is thus a need for a surgical instrument transport tray enclosure that reduces the number of cuts, holes and tears by protecting the wrap from shearing stress and motion.

### SUMMARY OF THE INVENTION

A surgical instrument tray enclosure comprising at least four sidewalls, at least four corners including pillars connected at exterior portion of the at least four corners of the at least four sidewalls and an interior corner having a rounded curve that is tangential to the pillars, the pillars are cylindrical in shape having bulbous top and bottom ends and extend a given distance beyond the bulbous top and bottom ends, and a base at a bottom of the at least four sidewalls to form a cavity where a wrapped surgical instrument tray may be inserted or bound within the cavity.

According to one embodiment, the at least four sidewalls may include windows. The base of the enclosure may also include at least two base openings. The surgical instrument tray enclosure can be comprised of an elastic material wherein the elastic material is selected from a group consisting of silicone, rubber, soft plastic, and flexible polyvinyl chloride. In one embodiment, the diameters of the pillars extend horizontally beyond a given distance of a given sidewall. The pillars may also include heights that extend vertically beyond top and bottom sides of the at least four sidewalls.

### BRIEF DESCRIPTION OF THE DRAWINGS

The invention is illustrated in the figures of the accompanying drawings which are meant to be exemplary and not limiting, in which like references are intended to refer to like or corresponding parts, and in which:

FIG. 1 illustrates a perspective view of a surgical instrument tray enclosure according to a first embodiment of the present invention;

FIG. 2 illustrates a top view of the surgical instrument tray enclosure according to the first embodiment of the present invention;

FIG. 3 illustrates a left or right side view of the surgical instrument tray enclosure according to the first embodiment of the present invention;

FIG. 4 illustrates a front or back view of the surgical instrument tray enclosure according to the first embodiment of the present invention;

FIG. 5 illustrates a perspective view of a surgical instrument tray enclosure according to a second embodiment of the present invention;

FIG. 6 illustrates a top view of the surgical instrument tray enclosure according to the second embodiment of the present invention;

FIG. 7 illustrates a left or right side view of the surgical instrument tray enclosure according to the second embodiment of the present invention; and

FIG. 8 illustrates a front or back view of the surgical instrument tray enclosure according to the second embodiment of the present invention;

FIG. 9 illustrates a perspective view of a surgical instrument tray enclosure according to a third embodiment of the present invention; and

FIG. 10 illustrates a side view of the surgical instrument tray enclosure according to a third embodiment of the present invention.

### DETAILED DESCRIPTION OF THE INVENTION

Subject matter will now be described more fully hereinafter with reference to the accompanying drawings, which form a part hereof, and which show, by way of illustration, exemplary embodiments in which the invention may be practiced. Subject matter may, however, be embodied in a variety of different forms and, therefore, covered or claimed subject matter is intended to be construed as not being limited to any example embodiments set forth herein; example embodiments are provided merely to be illustrative. It is to be understood that other embodiments may be utilized and structural changes may be made without departing from the scope of the present invention Likewise, a reasonably broad scope for claimed or covered subject matter is intended. The following detailed description is, therefore, not intended to be taken in a limiting sense.

Throughout the specification and claims, terms may have nuanced meanings suggested or implied in context beyond an explicitly stated meaning Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of exemplary embodiments in whole or in part.

An enclosure is described herein for protecting wrapped sterilized instrument trays during storage and transport. The enclosure may comprise an elastic band or cover that can be wrapped around a surgical instrument tray (not illustrated) made from stainless steel. According to certain embodiments, dimensions of the enclosure may be approximately 10 inches in width by 20 inches in length by 6 inches in height. Additionally, the enclosure may well include the ability for any given dimension to be stretched up to approximately 20% in size and withstand heats of up to about 300 degrees Fahrenheit. Suitable materials for forming the enclosure are primary soft, flexible, and moldable materials such as silicon, rubber, soft plastics, or flexible polyvinyl chloride (PVC). The primary attributes are that the material chosen provides the requisite strength and durability while also being compatible with the sterilants and temperatures used during the sterilization, cleaning and decontamination processes.

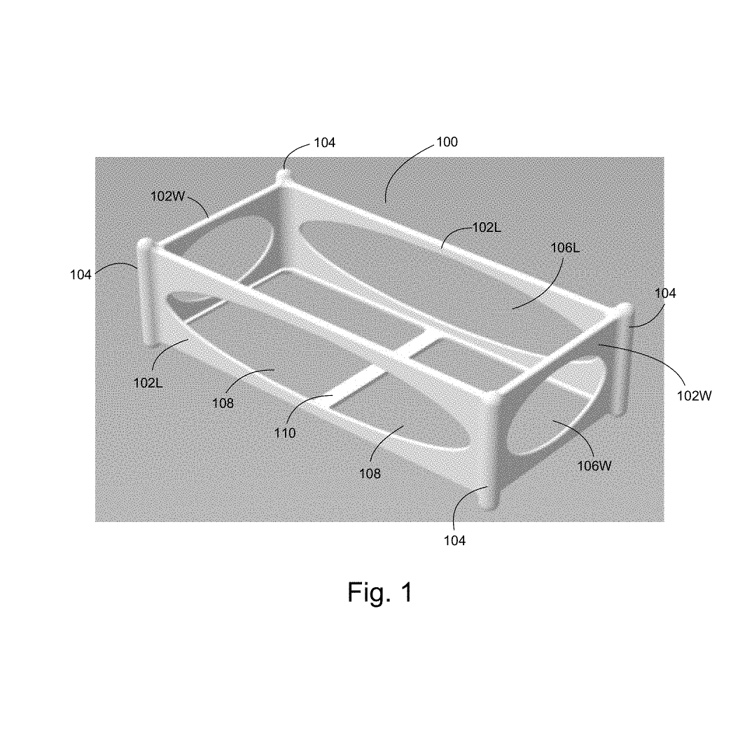

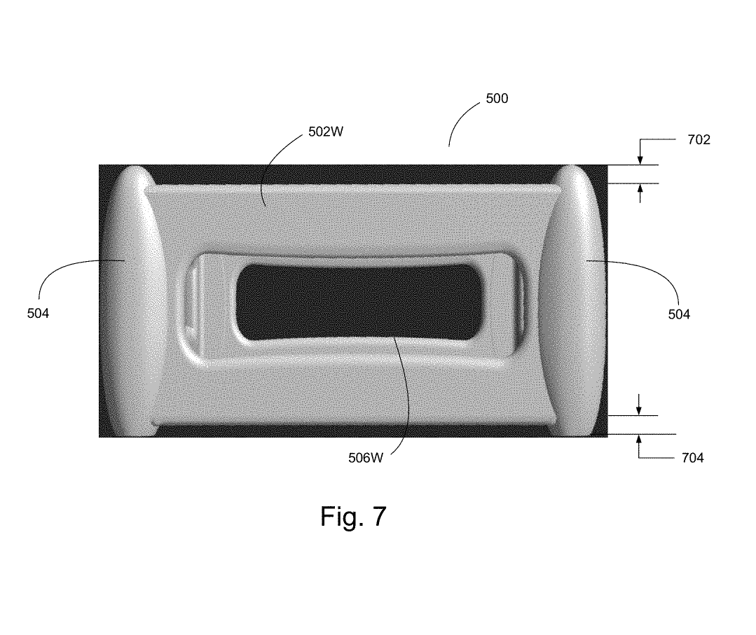

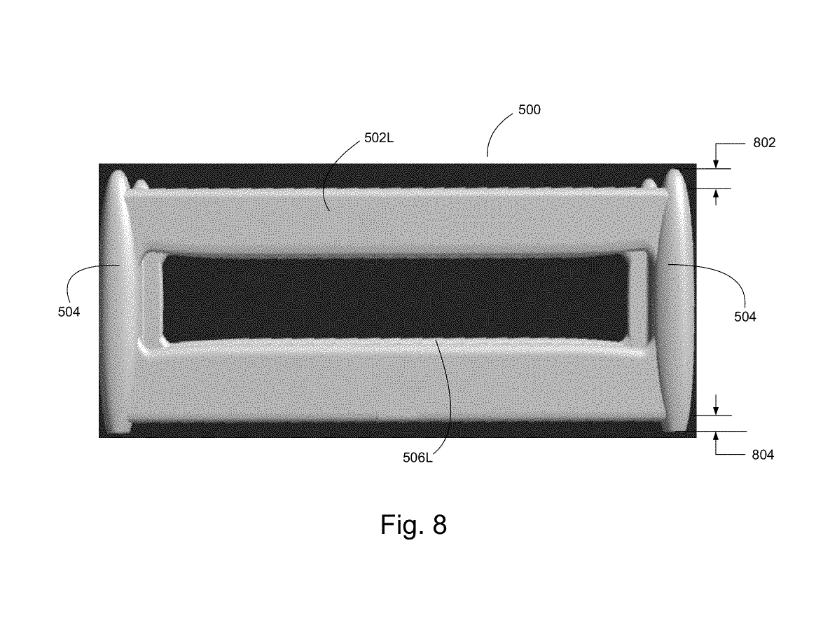

FIG. 1 through FIG. 4 present an enclosure for protecting wrapped sterilized instrument trays during storage and transport according to a first embodiment of the present invention. The enclosure 100 has at least four sides including sidewall 102L on each lengthwise side and sidewall 102W on each widthwise side. Pillars 104 join an exterior portion of the corners between sidewalls 102L and sidewalls 102W. A given sidewall may have a thickness of approximately half an inch. Sidewalls 102L include window 106L and sidewalls 102W include window 106W. The windows may be in the shape of an oval or circular shape. Alternative shapes of the windows may include a shape of a rectangle or a rectangle with rounded sides and corners such as illustrated in FIG. 7 and FIG. 8 according to a second embodiment of the present invention. Sidewall 102L includes a thickness 210 and sidewall 102W includes a thickness 212. Interior corner 202 comprises a union of an interior portion of sidewalls 102L and sidewalls 102W having a rounded curve that is tangential to pillar 104.

In use, instruments (not shown) needing to be sterilized are placed inside a tray and the combination is wrapped by a sterilization filter medium such as KIMGUARD® Sterile Wrap and SPUNGUARD® Sterilization Wrap materials from the Kimberly-Clark Corporation of Neenah, Wis. Once the sterilization process is complete, the sterilized trays are removed from the sterilization unit and then most typically stored on shelves (not shown) until needed. Once needed, the sterilized trays are taken from storage, transported to the operating room and then unwrapped. The instruments are removed from the tray and laid out for use. If the sterilization wrap is breached in any manner before unwrapping, the contents are considered contaminated and therefore must be rewrapped and sterilized again before the contents can be used. Enclosure 100 may be fitted, wrapped or stretched over a wrapped tray to provide for additional protection of the wrapped tray. According to another embodiment, the wrapped may be placed in a protective covering such as a bag made from Kevlar® ballistic material, or any other protective material, prior to fitment of enclosure 100. That is, enclosure 100 may be fitted over a Kevlar® bag containing a surgical tray wrapped in sterilization wrap.

According to the embodiment illustrated in FIG. 1 through FIG. 4, the pillars 104 are cylindrical in shape. Pillars 104 provide smooth rounded geometry at the sides, corners and ends (as opposed to sharp or pointed edges and corners) to reduce chances of puncturing the sterilization wrap of other surgical instrument trays during storage or transport. Additionally, pillars 104 include bulbous contours or geometry on the top and bottom ends having a diameter 208.

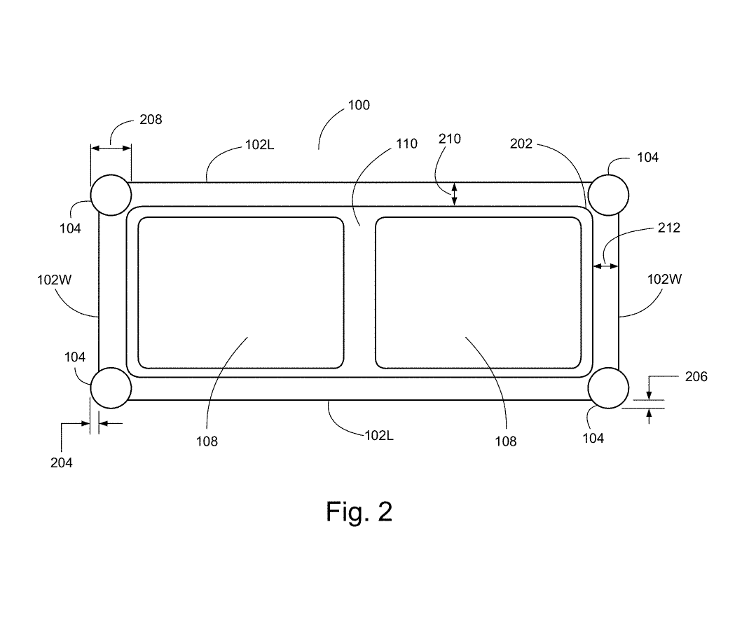

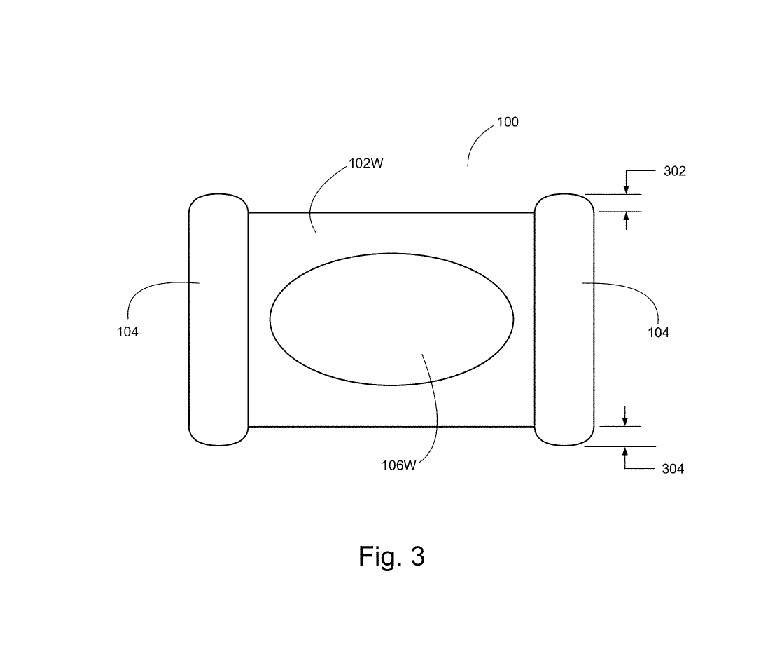

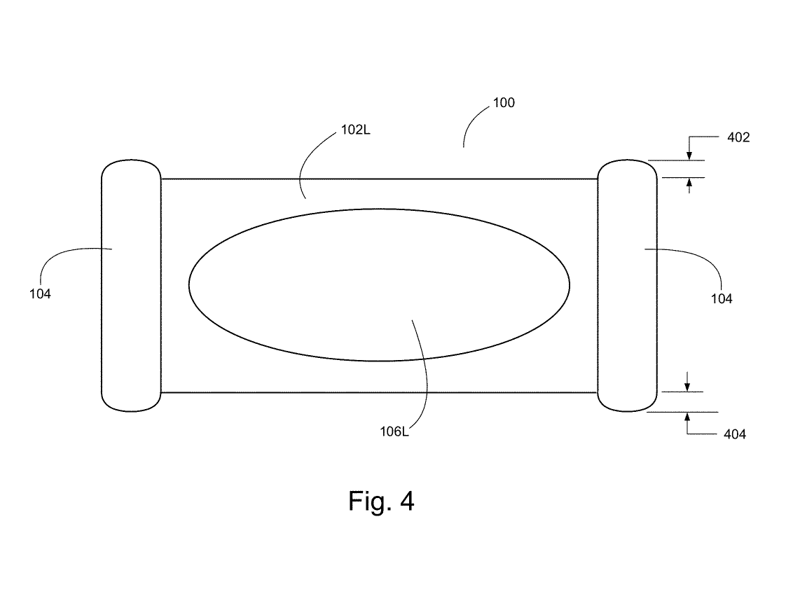

Referring to FIG. 2, on the lengthwise sides, diameter 208 of pillar 104 may extend horizontally beyond sidewall 102W a distance 204. Similarly, on the widthwise sides, diameter 208 of pillar 104 may extend horizontally beyond sidewall 102L a distance 206. Pillars 104 include heights that extend vertically beyond top and bottom sides of sidewall 102W and sidewall 102L. For example, FIG. 3 presents a side view of enclosure 100 including pillars 104 that are vertically longer than sidewall 102W by a distance 302 on the top end of pillars 104 and vertically longer than sidewall 102W by a distance 304 on the bottom end of pillars 104. FIG. 4 present a front or rear view of enclosure 100 including pillars 104 that are vertically longer than sidewall 102L by a distance 402 on the top end of pillars 104 and vertically longer than sidewall 102L by a distance 404 on the bottom end of pillars 104.

Enclosure 100 further includes a base 110 attached along on a bottom of sidewalls 102L, or alternatively, sidewalls 102W, and above a distance 304 or distance 404 from the bottom of pillars 104 to form a cavity. A wrapped surgical instrument tray may be inserted and fitted through the top of enclosure 100 to sit flush with base 110. The enclosure may be stretched to accommodate the surgical instrument tray within the cavity. Base 110 includes a pair of base openings 108. The base openings 108 may be rectangular in shape with rounded corners.

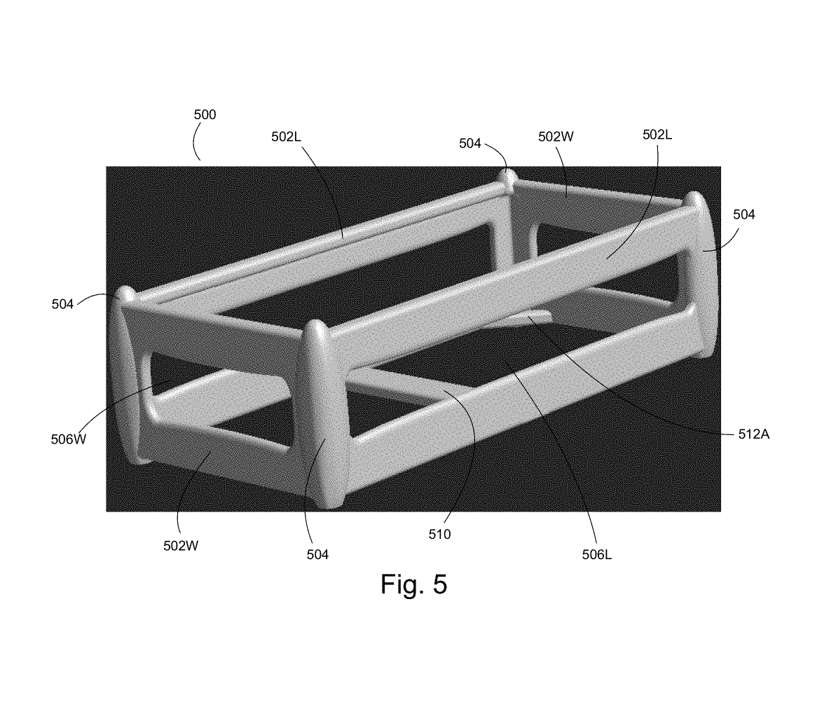

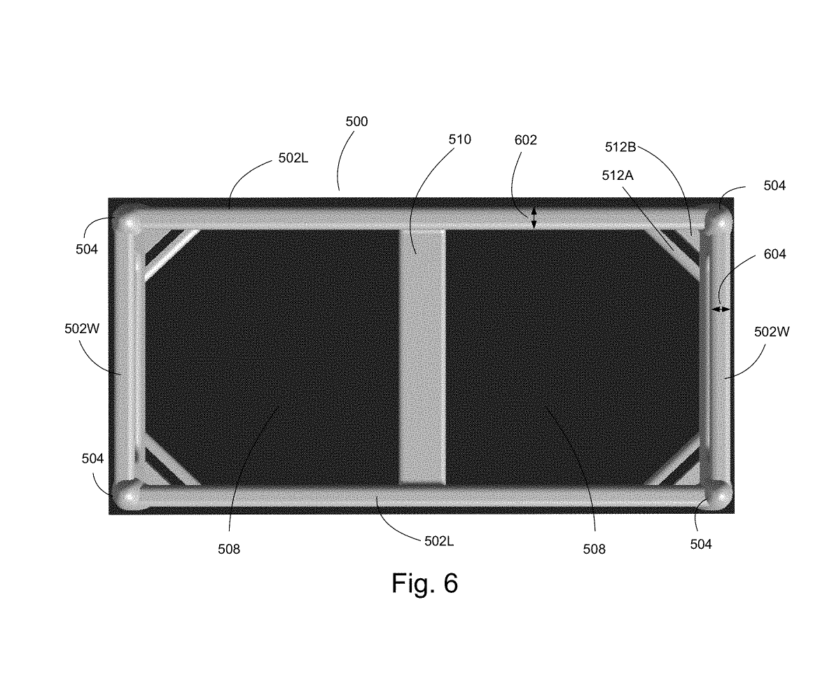

FIG. 5 through FIG. 8 present an enclosure for protecting wrapped sterilized instrument trays during storage and transport according to a second embodiment of the present invention. Enclosure 500 includes pillars 504 that are elliptical in shape including bulbous contours or geometry on the top ends and are flat on the bottoms ends. The enclosure 500 has at least four sides including sidewall 502L on each lengthwise side and sidewall 502W on each widthwise side. Pillars 504 join the corners between sidewalls 502L and sidewalls 502W. A given sidewall may have a thickness of approximately half an inch. Sidewalls 502L include window 506L and sidewalls 502W include window 506W. The windows may include a shape of a rectangle or a rectangle with rounded sides and corners such as illustrated in FIG. 7 and FIG. 8. Sidewall 502L includes a thickness 602 and sidewall 502W includes a thickness 604.

Referring to FIG. 7 and FIG. 8, pillars 504 include heights that extend vertically beyond top and bottom sides of sidewall 502W and sidewall 502L. For example, FIG. 7 presents a side view of enclosure 500 including pillars 504 that are vertically longer than sidewall 502W by a distance 702 on the top end of pillars 504 and vertically longer than sidewall 502W by a distance 704 on the bottom end of pillars 504. FIG. 8 present a front or rear view of enclosure 500 including pillars 504 that are vertically longer than sidewall 502L by a distance 802 on the top end of pillars 504 and vertically longer than sidewall 502L by a distance 804 on the bottom end of pillars 504.

Enclosure 500 further includes a base 510 and corner base structures 512A and 512B. Base 510 may be attached along a bottom portion of sidewalls 502L, or alternatively sidewalls 502W, and above a distance 704 or distance 804 from the bottom of pillars 504 to form a cavity. Corner base structures 512A and 512B may be attached between sidewalls 502L and sidewalls 502W on each corner above a distance 704 or distance 804 from the bottom of pillars 504. Base 510 includes a pair of base openings 508. The base openings 508 may be rectangular in shape with rounded corners.

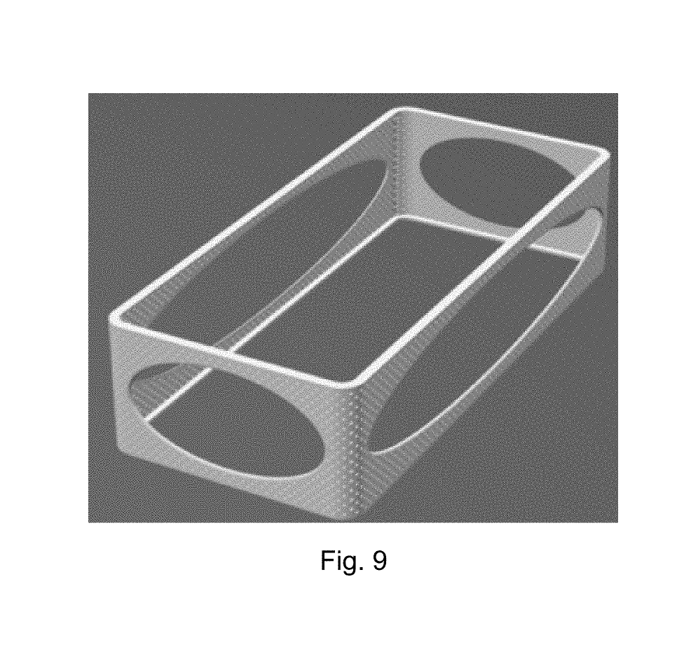

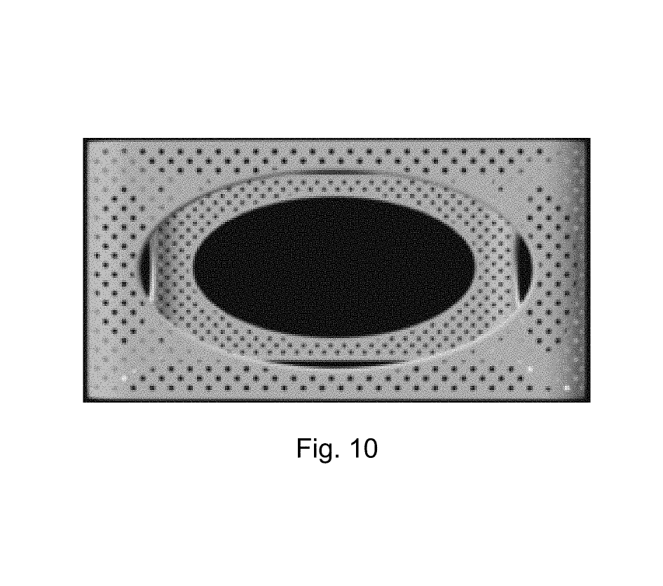

FIGS. 9 and 10 present an enclosure for protecting wrapped sterilized instrument trays during storage and transport according to a third embodiment of the present invention where the enclosure includes perforations.

FIGS. 1 through 10 are conceptual illustrations allowing for an explanation of the present invention. Notably, the figures and examples above are not meant to limit the scope of the present invention to a single embodiment, as other embodiments are possible by way of interchange of some or all of the described or illustrated elements. Moreover, where certain elements of the present invention can be partially or fully implemented using known components, only those portions of such known components that are necessary for an understanding of the present invention are described, and detailed descriptions of other portions of such known components are omitted so as not to obscure the invention. In the present specification, an embodiment showing a singular component should not necessarily be limited to other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present invention encompasses present and future known equivalents to the known components referred to herein by way of illustration.

The foregoing description of the specific embodiments will so fully reveal the general nature of the invention that others can, by applying knowledge within the skill of the relevant art(s) (including the contents of the documents cited and incorporated by reference herein), readily modify and/or adapt for various applications such specific embodiments, without undue experimentation, without departing from the general concept of the present invention. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

What is claimed is:

1. A surgical instrument tray enclosure comprising:
at least four sidewalls;
at least four corners including at least four pillars connected at exterior portion of the at least four corners of the at least four sidewalls and an interior corner having a rounded curve that is tangential to the pillars, the pillars are cylindrical in shape having bulbous top and bottom ends and extend a given distance beyond the bulbous top and bottom ends; and
a base at a bottom of the at least four sidewalls to form a cavity where a wrapped surgical instrument tray may be inserted or bound within the cavity.

2. The surgical instrument tray enclosure of claim 1 wherein the at least four sidewalls include windows.

3. The surgical instrument tray enclosure of claim 1 wherein the base includes at least two base openings.

4. The surgical instrument tray enclosure of claim 1 wherein the surgical instrument tray enclosure is comprised of an elastic material.

5. The surgical instrument tray enclosure of claim 4 wherein the elastic material is selected from a group consisting of silicone, rubber, soft plastic, and flexible polyvinyl chloride.

6. The surgical instrument tray enclosure of claim 1 wherein diameters of the pillars extend horizontally beyond a given distance of a given sidewall.

7. The surgical instrument tray enclosure of claim 1 wherein the pillars include heights that extend vertically beyond top and bottom sides of the at least four sidewalls.
